(19) United States
(12) Patent Application Publication
(10) Pub. No.: US 2003/0073945 A1
(43) Pub. Date: Apr. 17, 2003

(54) Ozone hemodiafiltration device

(76) Inventor: Liang-Kuang Diang, Taipei Hsien (TW)

Correspondence Address: RABIN & BERDO, P.C., Suite 500, 1101 14th Street, N.W., Washington, DC, 20005, US

(21) Appl. No.: 09/977,966

(22) Filed: Oct. 17, 2001

(51) Int. Cl. A61M 37/00; C02F 1/44; B01D 63/00

(52) U.S. Cl. 604/5.01; 210/646; 210/321.71

(57) ABSTRACT

An ozone hemodiafiltration device comprising a dialysate fluid circuit, extracorporeal circuit for blood and a replenishing solute circuit. The dialysate fluid circuit provides aseptic isotonic solution with mineral and nutrients in specific ratio to a dialyzer. The extracorporeal circuit for blood draws blood flow from human bodies, passes through the dialyzer, and turns back to human bodies. The replenishing solute circuit conducts the isotonic solution from the dialysate fluid circuit into a filter for re-filtration and the re-filtered isotonic solution is injected into human bodies through the extracorporeal circuit for blood. The device features on an ozone (O₃) generator, which converts the reverse osmosis water into ozone water solution, disposed before the water inlet end of the dialysate fluid circuit. By controlling the flow of ozone water solution into the extracorporeal circuit for blood and the replenishing solute circuit, the ozone in blood keeps at 3-6 ppm concentrations.

### BACKGROUND OF THE INVENTION

1. Field of the Invention

The present invention relates to a device for ozone hemodiafiltration, especially to a device that injects ozone into the human body safely and smoothly so as to kill microorganisms such as bacteria and virus.

2. Description of the Prior Art

Accordingly, ozone generally exists in the gas form and is a kind of most efficient bacteriacide, decade times powerful than other disinfectant drugs. The chemical character of ozone is unstable thus it is easily to combine with other molecules, oxidizes other substance and itself is reduced into oxygen, which is harmless substance. Therefore, ozone is the most evaluated disinfectant, which has a flash and efficient effect on killing bacteria or other microorganisms without the problem of residual.

Due to the character of easily combining with other molecules, ozone currently is used for cleaning and deodorization of air. By ozonation, passing ozone through water, ozone is used for disinfection of apparatus or green food, freshening food, removing pesticide. Act as a disinfectant, ozone is often only for external use such as broken wound on skin, haven't applied on internal use of human bodies. However, the abuse of antibiotics causes more stains of drug-resistant bacteria. Thus more and more diseases are difficult to be eradicated. Therefore, there is an urgent need to apply ozone to internal use of human bodies since ozone is an extremely powerful biocide against most kinds of microorganisms.

### SUMMARY OF THE INVENTION

It is therefore a primary object of the present invention to provide an ozone hemodiafiltration device which passes ozone into the human bodies as a powerful disinfectant without the problem of residual so as to cure people of diseases caused by bacteria, virus or even cancer cells.

### BRIEF DESCRIPTION OF THE DRAWINGS

The accomplishment of the above-mentioned object of the present invention will become apparent from the following description and its accompanying drawings which disclose illustrative an embodiment of the present invention, and are as follows:

FIG. 1 is a schematic diagram of circuits of an ozone hemodiafiltration device in accordance with the present invention.

### DETAILED DESCRIPTION OF THE PREFERRED EMBODIMENT

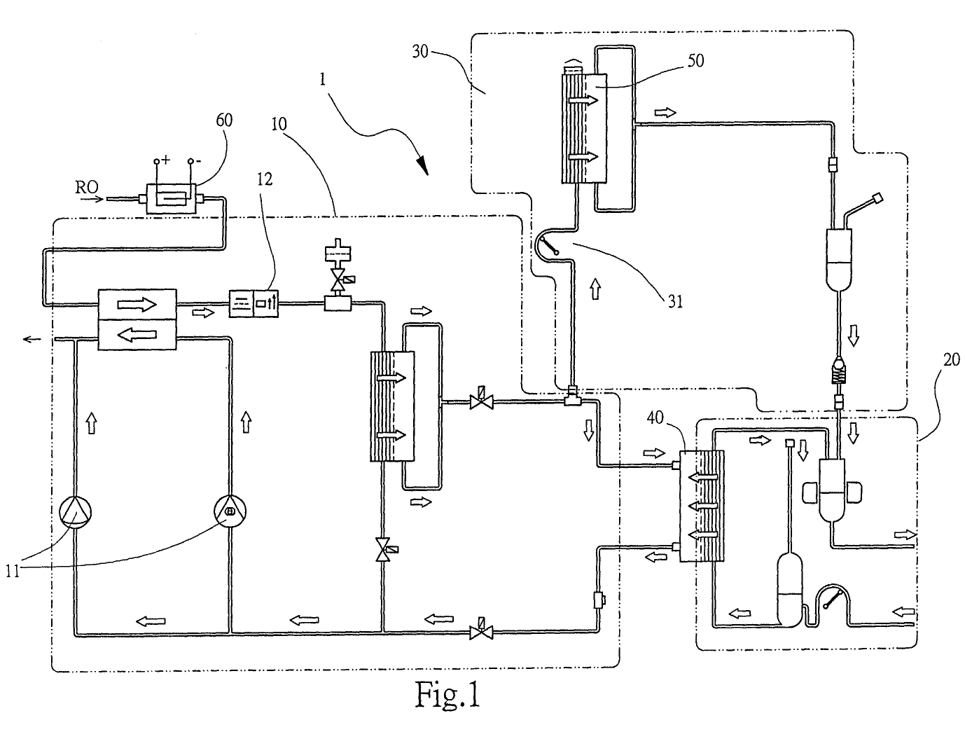

Refer to FIG. 1, the present invention includes ozone hemodiafiltration device 1 having a dialysate fluid circuit 10, extracorporeal circuit for blood 20 and a replenishing solute circuit 30. The function of the dialysate fluid circuit 10 is to provide aseptic isotonic solution into a dialyzer (artificial kidney) 40. The isotonic solution is composed of mixture of reverse osmosis (RO) water, mineral and chemical concentrate in specific ratio. The isotonic solution is conducted to dialyzer 40 and replaces continually for efficient removing wastes by solute diffusion and then is discharged by a pump 11.

The extracorporeal circuit for blood 20 draws the blood flow from human body, passing through the dialyzer 40 then turn back to human blood vessels.

The replenishing solute circuit 30 is to conduct the isotonic solution 10 through a filter 50 for re-filtration and then the isotonic solution 10 is transported directly into the extracorporeal circuit for blood 20 and enters the human bodies for water supplement.

The feature of the present invention is that an ozone (O₃) generator 60 is disposed before the water inlet end of the dialysate fluid circuit 10. The ozone (O₃) generator 60 is used to convert the reverse osmosis water into ozone water solution and then the ozone aqueous is mixed with the isotonic solution. The flow of the isotonic solution with aqueous ozone is under control and is continuously injected into human bodies through the replenishing solute circuit 30 and the extracorporeal circuit for blood 20. The ozone in blood of patient keeps at 3-6 ppm concentrations and flows around the whole body for long-term effect against microorganisms. For precisely monitoring the concentration of ozone in blood, a monitor 12 is installed at the water inlet end of the ozone water solution for monitoring the ozone concentration momently for reference of adjusting the flow of pump 31. Thus the ozone concentration in human bodies can be easily and precisely controlled at 3-6 ppm concentrations.

Although ozone is easy to breakdown into water and oxygen or reduced by other substance into oxygen during several minutes or seconds, a therapy circle of the present invention—hemodialysis machine 1 takes at least 2-4 hours and during this period the present invention provides adequate amount of ozone continuously so as to keep certain concentrations of ozone. Sufficient time for reaction and stable concentrations of ozone, both make ozone effectively kill microorganisms such as bacteria, virus and cancer cell inside human bodies.

It should be noted that the above description and accompanying drawings are only used to illustrated some embodiments of the present invention, not intended to limit the scope thereof. Any modification of the embodiments should fall within the scope of the present invention.

What is claimed is:

1. An ozone hemodiafiltration device comprising a dialysate fluid circuit, extracorporeal circuit for blood and a replenishing solute circuit, wherein said dialysate fluid circuit is used to provide aseptic isotonic solution into a dialyzer (artificial kidney); said extracorporeal circuit for blood draws blood flow from human blood vessels, passing through the dialyzer and then turns back to human bodies; said replenishing solute circuit conducts the isotonic solution through a filter for re-filtration so that the isotonic solution can be transported into the extracorporeal circuit for blood and enters human bodies for water supplement; the present invention is characterized in that an ozone (O₃) generator is disposed on before the water inlet end of said dialysate fluid circuit for converting the reverse osmosis water into ozone water solution, which is mixed with the isotonic solution and inject into human bodies through said replenishing solute circuit and said extracorporeal circuit for blood in order to kill microorganisms such as bacteria, virus or cancer cells; the ozone in blood is at 3-6 ppm concentrations.

2. An ozone hemodiafiltration device as claimed in claim 1, wherein a monitor is disposed on the ozone water input end for monitoring ozone concentrations.
